(19) United States
(12) Patent Application Publication
(10) Pub. No.: US 2013/0035681 A1
(43) Pub. Date: Feb. 7, 2013

(54) NOVEL CATHETER FOR CONTIGUOUS RF ABLATION

(71) Applicants: Raj SUBRAMANIAM, Fremont, CA (US); Zaya TUN, Livermore, CA (US); Kurt SPARKS, San Carlos, CA (US)

(72) Inventors: Raj SUBRAMANIAM, Fremont, CA (US); Zaya TUN, Livermore, CA (US); Kurt SPARKS, San Carlos, CA (US)

(73) Assignee: BOSTON SCIENTIFIC SCIMED, INC., Maple Grove, MN (US)

(21) Appl. No.: 13/564,539

(22) Filed: Aug. 1, 2012

Related U.S. Application Data
(60) Provisional application No. 61/515,238, filed on Aug. 4, 2011.

(51) Int. Cl. A61B 18/18

(52) U.S. Cl. 606/33

(57) ABSTRACT

A tissue ablation device comprising an elongate member having a proximal end, a distal end and a side wall defining a lumen, and an elongate ablation member having an ablation element proximate a distal end thereof, the elongate ablation member rotatable about an longitudinal axis of the elongate member, and wherein one of the elongate ablation member and the elongate member is at least partially contained within the other and methods of use therefor.

### CROSS-REFERENCE TO RELATED APPLICATION

This application claims the benefit of U.S. Provisional Application Ser. No. 61/515,238, filed Aug. 4, 2011, the entire disclosure of which is incorporated herein by reference.

### TECHNICAL FIELD

The invention generally pertains to structures for intravascular nerve modulation and ablation therapies, and methods of use thereof

### BACKGROUND

Certain treatments require the temporary or permanent interruption or modification of select nerve function. One example treatment is pulmonary vein isolation which is sometimes used to treat conditions related to paroxysmal atrial fibrillation. Ablation employing one or more ring electrodes may reduce or eliminate this fibrilation, which may provide a corresponding reduction in the associated undesired symptoms.

Atrial fibrillation (AFib) is believed to be the result of the simultaneous occurrence of multiple wavelets of functional re-entry of electrical impulses within the atria, resulting in a condition in which the transmission of electrical activity becomes so disorganized that the atria contracts irregularly. Once considered a benign disorder, AFib now is widely recognized as the cause of significant morbidity and mortality. The most dangerous outcome from AFib is thromboembolism and stroke risk, the latter due to the chaotic contractions of the atria causing blood to pool. This in turn can lead to clot formation and the potential for an embolic stroke. According to data from the American Heart Association, about 75,000 strokes per year are AFib-related.

Some radiofrequency (RF) ablation protocols that have been proven to be highly effective in tachycardia treatment while exposing a patient to minimal side effects and risks. Radiofrequency catheter ablation is generally performed after conducting an initial mapping study where the locations of the arrhythmogenic site and/or accessory pathway are determined. After a mapping study, an ablation catheter is usually introduced to the target heart chamber and is manipulated so that the ablations tip electrode lies exactly at the target tissue site. Radiofrequency energy or other suitable energy is then applied through the tip electrode to the cardiac tissue in order to ablate the tissue of the arrhythmogenic site or the accessory pathway. By successfully destroying that tissue, the abnormal signal patterns responsible for the tachycardia may be eliminated. However, in the case of atrial fibrillation (AFib) or atrial flutter, multiple arrhythmogenic sites and/or multiple accessory pathways exist. The conventional catheter with a single “stationary” ablation electrode cannot effectively cure the symptoms. In the case of paroxysmal atrial fibrillation, a circular lesion at about the pulmonary vein is required. Prior art devices have attempted to achieve a circular lesion by employing a plurality of spot ablations; however this approach may leave conduction pathways in the gaps between spots. Other devices have used a circular electrode associated with a balloon surface which may result in inconsistent contact and ablation.

### SUMMARY

It is desirable to provide an improved and/or alternative catheter capable of producing more uniform circular ablation.

Some embodiments pertain to a tissue ablation device, comprising an elongate member having a proximal end, a distal end, and a non-occlusive anchoring member proximate the distal end thereof. The non-occlusive anchoring member may be configured to removably anchor the elongate member within a lumen of a patient. The device may also include a tubular member having an opening extending through a side wall of the tubular member near the distal end of the tubular member. The device may also include an elongate ablation member having an ablation element at the distal end thereof. The elongate member may extend distally out the tubular member and the elongate ablation member may extend out through the opening in the side wall of the tubular member. When the non-occlusive anchoring member has fixed the elongate member within a lumen of a patient, the tubular member may rotate about the elongate member such that the elongate ablation member extending through the second opening may circumnavigate the elongate member.

The elongate ablation member may include one or more electromyographic sensors. The elongate ablation member may have a first curve proximate the distal end thereof and may further include a second curve proximate the distal end thereof, the second curve curving in a direction generally opposite the first curve. The elongate member may include a lumen. The non-occlusive anchoring member may be self-expanding and/or may be actuatable between a collapsed configuration and an expanded configuration. For example, the non-occlusive anchoring member may be a balloon or may comprise two or more struts. The ablation element may be an electrode or may comprise a laser or other ablation element. The non-occlusive anchoring member may be rotatable relative to the elongate member. The non-occlusive anchoring member may be disposed on a Tuohy-Borst adapter.

In some embodiments, the tissue ablation may comprise a mapping system capable of mapping electrical activity detected by the one or more electromyographic sensors, and may further comprise a multi-axis computerized drive adapted to trace a pre-established ablation line.

Some embodiments pertain to a tissue ablation device comprising an elongate member having a proximal end, a distal end and a side wall defining a lumen, and an elongate ablation member having an ablation element proximate a distal end thereof, the elongate ablation member rotatable about an longitudinal axis of the elongate member, and wherein one of the elongate ablation member and the elongate member is at least partially contained within the other. The elongate ablation member may be movable longitudinally to vary a radial distance between the elongate member and the ablation element. The distal end of the elongate ablation member may be proximal the distal end of the elongate member. The elongate member may further comprise an anchoring member proximate the distal end thereof. The anchoring member may be non-occlusive, and may be actuatable between a collapsed configuration and an expanded configuration. The anchoring member may, for example, be a balloon or may comprise two or more struts. The anchoring member may be capable of rotation relative to the elongate member. The ablation element may be an electrode and may further comprise one or more electromyographic sensors. The ablation element may comprise a laser. The tissue ablation device may further comprise a mapping system capable of mapping electrical activity detected by the one or more eletromyographic sensor, and may further comprise a multi-axis computerized drive adapted to trace a pre-established ablation line. The elongate ablation member may be capable of circumnavigating the elongated member.

Some embodiments pertain to a method of ablating tissue that may comprise the steps of positioning a tissue ablating device, for example such as described above, such that the distal portion of the elongate member is within a body lumen, positioning the elongate ablation member adjacent to body tissue surrounding the body lumen, activating the ablation element at the distal end of the elongate ablation member to ablate tissue adjacent to the ablation element; and rotating the elongate ablation member around the elongate member such that the activated ablation element circumnavigates the elongate member while continuously ablating tissue adjacent to the ablation element. The step of positioning a tissue ablating device may be such that the distal portion of the elongate member is within a body lumen includes positioning and expanding an anchoring member within the body lumen thereby removably fixing the elongate member relative to the body lumen. After the step of positioning the elongate ablation member adjacent to body tissue surrounding the body lumen and prior to activating the ablation element a system capable of mapping electrical activity detected by one or more electromyographic sensors may map said electrical activity. The step of rotating the elongate ablation member around the elongate member such that the activated ablation element circumnavigates the elongate member while continuously ablating tissue adjacent to the ablation element may be directed and controlled by a multi-axis computerized drive adapted to trace a pre-established ablation line. The pre-established ablation line traced by the activated ablation element under the direction and control of the multi-axis computerized drive adapted to trace a pre-established ablation line may be determined by a map generated by a system capable of mapping electrical activity detected by one or more electromyographic sensors.

### BRIEF DESCRIPTION OF DRAWINGS

FIGS. 1A and 1B illustrate, respectively, the proximal and distal portions of a tissue ablation device according to the invention.

FIG. 2 illustrates the distal portion of a tissue ablation device according to the invention.

FIG. 3 illustrates an end view of a tissue ablation device in situ.

### DETAILED DESCRIPTION

The following description should be read with reference to the drawings wherein like reference numerals indicate like elements throughout the several views. The drawings, which are not necessarily to scale, are not intended to limit the scope of the claimed invention. The detailed description and drawings illustrate example embodiments of the claimed invention.

All numbers are herein assumed to be modified by the term “about.” The recitation of numerical ranges by endpoints includes all numbers subsumed within that range (e.g., 1 to 5 includes 1, 1.5, 2, 2.75, 3, 3.80, 4, and 5).

As used in this specification and the appended claims, the singular forms “a”, “an”, and “the” include the plural referents unless the content clearly dictates otherwise. As used in this specification and the appended claims, the term “or” is generally employed in its sense including “and/or” unless the content clearly dictates otherwise.

It is noted that references in the specification to “an embodiment”, “some embodiments”, “other embodiments”, etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to effect such feature, structure, or characteristic in connection with other embodiments whether or not explicitly described unless cleared stated to the contrary.

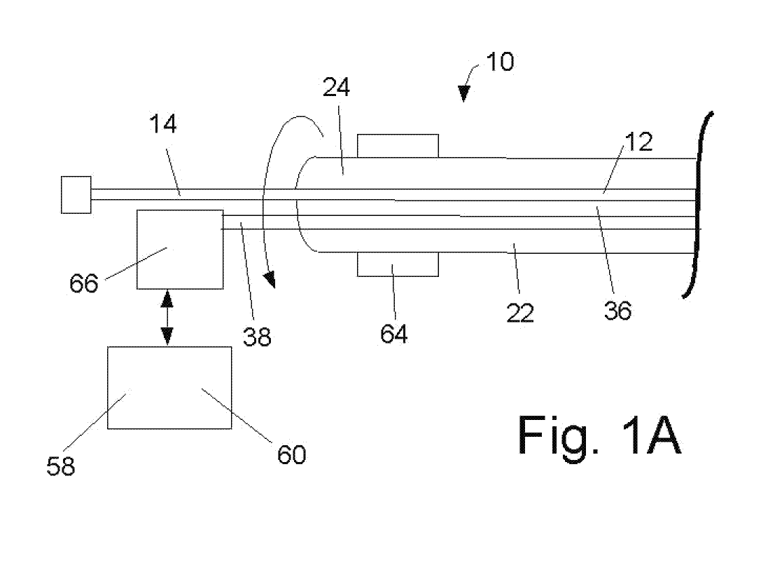

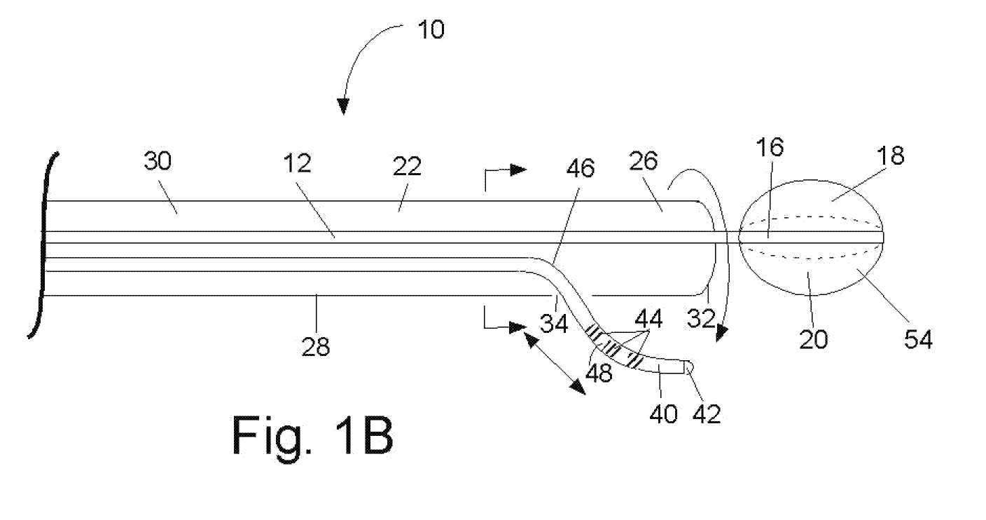

FIGS. 1A and 1B show, respectively, the proximal and distal portions of a tissue ablation device 10. The tissue ablation device 10 includes an elongate member 12 having a proximal end 14, a distal end 16, and a fixing member 18 configured to removably fix the elongate member within the lumen of a patient. The fixing member 18 in this embodiment is a non-occlusive member 20, and is illustrated as a multi-lobed balloon. The tissue ablation device 10 further includes a tubular member 22 having a proximal end 24, a distal end 26, a side wall 28 defining a lumen 30 extending to the distal end 26, a first opening 32 at the distal end 26 of the tubular member 22 and a second opening 34, the second opening 34 extending through the side wall 28 of the tubular member 22 proximate and proximal the distal end 26 of the tubular member 22. The tissue ablation device 10 further includes an elongate ablation member 36 having a proximal end 38, a distal end 40 and an ablation element 42 at the distal end 40. The elongate member 12 extends through the lumen 30 of the tubular member 22 and is extensible through the first opening 32 and movable longitudinally and rotably with respect to the tubular member 22. The elongate ablation member 36 extends through the second opening 34 and is movable longitudinally and rotably with respect to the tubular member 22. The tissue ablation device 10 is configured such that when the fixing member 18 has been expanded to fix the elongate member 12 within and with respect to the lumen of a patient, the tubular member 22 may rotate about the elongate member 12 such that the elongate ablation member 36 may circumnavigate the elongate member 12. The elongate member 12 and the elongate ablation member 36 may also be movable with respect to each other. Moving the elongate ablation member 36 in the tubular member 22 may change the radial distance between the ablation element 42 and the elongate member 12.

The ablation element 42 may be an electrode, a laser or other suitable ablation element such as a cryogenic ablation element. The elongate ablation member 36 may include one or more sensors 44 proximate and proximal the distal end 40. These sensors 44 may be electromygraphic sensors or other suitable sensors. The elongate ablation member may have a first curve 46 proximate the distal end 40 and may also include a second curve 48, also proximate the distal end 40, but curving in a direction opposite to that of the first curve 46. If an electrode, the electrode may be formed from any suitable material such as, but not limited to platinum, gold, stainless steel, cobalt alloys, or other non-oxidizing materials. In some instances, titanium, tantalum, or tungsten may be used.

It is contemplated that the electrode may take any shape desired, such as, but not limited to, square, rectangular, circular, oblong, etc. In some embodiments, the electrode may have rounded edges in order to reduce the affects of sharp edges on current density. In some instances, the electrodes may have an aspect ratio of 2:1 (length to width).

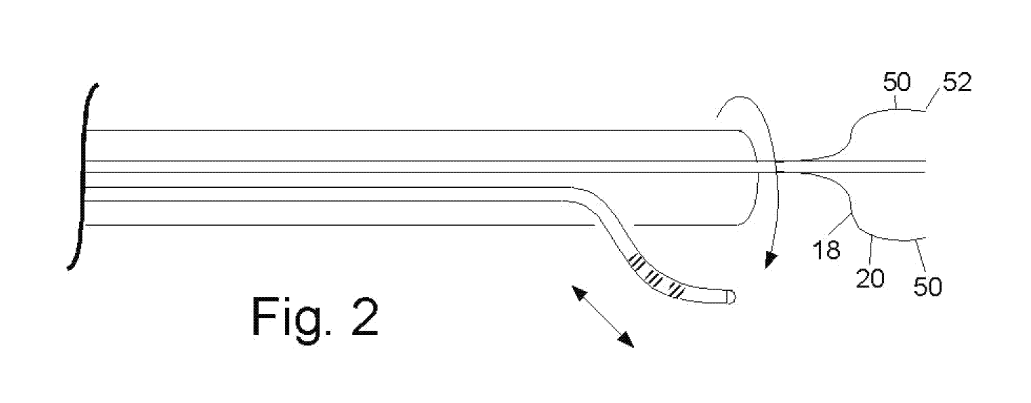

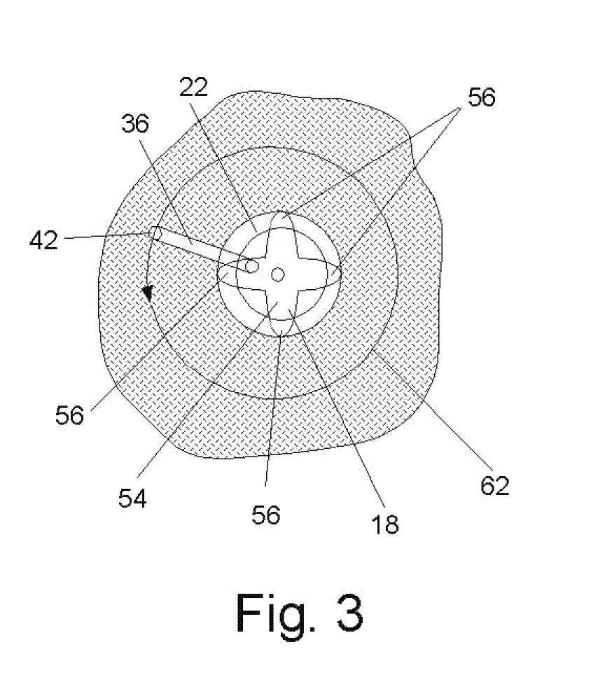

The elongate member 12 may include one or more lumens (not shown). Suitable lumens may include an inflation lumen or a guidewire lumen. The fixing member 18 may be a self expanding member and may be a self expanding non-occlusive member 20. A self-expanding non-occlusive member may include one or more struts 50 as shown in FIG. 2 to form a self-expanding non-occlusive member 52. The fixing member 18 may be actuatable between a collapsed configuration and an expanded configuration. The fixing members 18 illustrated in FIGS. 1A and 2 are both in an expanded configuration. The fixing member may be biased to an expanded configuration or may be biased to a collapsed configuration. The fixing member may include a balloon 54 and the balloon may have one, two, three, four or more lobes 56 as best seen in the end view of FIG. 3. The fixing member 18 may be disposed on a Tuohy-Borst adapter.

A control 58 may be operatively connected to the tissue ablation system 10, including an operative connection to the elongate member 12 the tubular member 22 and the elongate ablation member 36. The control may be configured to operate ablation element 42. In some embodiments, the control 58 may be used to activate sensors 44 and to receive signals therefore. The control may comprise a mapping system capable of mapping the topology of the patient's system proximate to sensors 44. Rotating the elongate ablation member 36 about the elongate member 12 may allow the mapping system to map the region in the patient's system proximate to the distal portion of the tissue ablation device 10. The sensors may include one or more electromyographic sensors and may therefore be capable of mapping electrical activity in the patient's system.

The tissue ablation system may further include a multi-axis computerized drive 60 that can move the ablation element 42 to trace a predetermined ablation line 62. Such an ablation line may be circular as shown in FIG. 3 or may be substantially linear or may include circular and linear elements. The multi-axis computerized drive 60 may include an actuator 64 on the tubular member 22 that can move the tubular member 22 rotationally and longitudinally and may include an actuator 66 on the elongate ablation element 36 that can move the elongate ablation element 36 rotationally and longitudinally.

In use, the tissue ablation system is positioned within a body lumen such as a pulmonary vein. A fixing element 18 such as the non-occlusive element 20 may be activated to fixed the distal end 16 of the elongate member 12 within the body lumen. The elongate ablation member 36 is positioned adjacent to body tissue surrounding the body lumen and the ablation element 42 is activated and rotated to ablate tissue adjacent to the ablation element while circumnavigating the elongate member to provide in the body tissue a continuous line 62 of ablated tissue, which line substantially blocks electrical signals from passing through the line 62. The elongate ablation member 36 may be continuously longitudinally adjusted during the ablation procedure to ensure contact between the ablation element 42 and the body tissue.

In some example uses, sensors 44 are used to map the topology and/or electrical activity of the surrounding body tissue. This information may be used by the control 58 to activate a multi-axis computerized drive 60 to maintain contact between the ablation element 42 and the body tissue along a predetermined ablation path. The ablation element 42 may continuously ablate body tissue along the predetermined ablation path. The sensors may then be used to map electrical activity subsequent to ablation to determine the efficacy of the procedure. The fixing element 18 may be deactivated and the tissue ablation system may be withdrawn.

Various modifications and alterations of this invention will become apparent to those skilled in the art without departing from the scope and principles of this invention, and it should be understood that this invention is not to be unduly limited to the illustrative embodiments set forth hereinabove. All publications and patents are herein incorporated by reference to the same extent as if each individual publication or patent was specifically and individually indicated to be incorporated by reference.

What is claimed is:

1. A tissue ablation device, comprising:
an elongate member having a proximal end, a distal end, and a non-occlusive anchoring member proximate the distal end thereof, the non-occlusive anchoring member configured to removably anchor the elongate member within a patient;
a tubular member having a proximal end, a distal end, a side wall defining a lumen extending to the distal end, a first opening at the distal end of the tubular member and a second opening, the second opening extending through the side wall of the tubular member proximate and proximal the distal end of the tubular member; and
an elongate ablation member having a proximal end, a distal end, and an ablation element at the distal end thereof,
the elongate member extending through the lumen of the tubular member and extensible through the first opening,
the elongate ablation member extending and extensible through the second opening,
wherein when the non-occlusive anchoring member has anchored the elongate member within a patient, the tubular member may rotate about the elongate member such that the elongate ablation member distal end may orbit the elongate member so as to trace a closed loop about the elongate member.

2. The tissue ablation device of claim 1, wherein the elongate ablation member further comprises one or more electromyographic sensors.

3. The tissue ablation device of any of claims 1-2, wherein the elongate ablation member has a first curve proximate the distal end thereof.

4. The tissue ablation device of claim 3, wherein the, wherein the elongate ablation member has a second curve proximate the distal end thereof, the second curve curving in a direction generally opposite the first curve.

5. The tissue ablation device of claim 1 wherein the non-occlusive anchoring member is actuatable between a collapsed configuration and an expanded configuration.

6. The tissue ablation device of claim 6, wherein the non-occlusive anchoring member comprises two or more struts.

7. The tissue ablation device of claim 1, wherein the ablation element is an electrode.

8. The tissue ablation device of claim 1, wherein the non-occlusive anchoring member is rotatable relative to the elongate member.

9. The tissue ablation device of 1, further comprising a mapping system capable of mapping electrical activity detected by the one or more electromyographic sensors.

10. The tissue ablation device of claim 1, wherein the elongate ablation member further comprises a multi-axis computerized drive adapted to trace a pre-established ablation line.

11. A tissue ablation device comprising:
an elongate member having a proximal end, a distal end and a side wall defining a lumen; and
an elongate ablation member having an ablation element proximate a distal end thereof, the elongate ablation member rotatable about an longitudinal axis of the elongate member, and wherein one of the elongate ablation member and the elongate member is at least partially contained within the other.

12. The tissue ablation device of claim 16, wherein the elongate ablation member is movable longitudinally to vary a radial distance between the elongate member and the ablation element.

13. The tissue ablation device of claim 16, wherein the distal end of the elongate ablation member is proximal the distal end of the elongate member.

14. The tissue ablation device of claim 16, wherein the elongate member further comprises an anchoring member proximate the distal end thereof.

15. The tissue ablation device of claim 14, wherein the anchoring member is non-occlusive.

16. The tissue ablation device of claim 19, wherein the anchoring member is capable of rotation relative to the elongate member.

17. The tissue ablation device of claim 16, wherein the ablation member further comprises one or more electromyographic sensors

18. The tissue ablation device of claim 16, further comprising a mapping system capable of mapping electrical activity detected by the one or more electromyographic sensors.

19. The tissue ablation device of claim 16, wherein the elongate ablation member further comprises a multi-axis computerized drive adapted to trace a pre-established ablation line.

20. The tissue ablation device of claim 16, wherein the elongate ablation member is capable of circumnavigating the elongated member.
